(19) United States
(12) Patent Application Publication
(10) Pub. No.: US 2026/0216691 A1
(43) Pub. Date: Jul. 30, 2026

(54) ZEOLITIC ADSORBENT FOR HIGH PRODUCTIVITY XYLENE SEPARATION

(71) Applicants: IFP ENERGIES NOUVELLES, Rueil-Malmaison (FR); ARKEMA FRANCE, Puteaux (FR)

(72) Inventors: Ludivine BOUVIER, Lacq (FR); Maria MANKO, Rueil-Malmaison (FR); Javier PEREZ-PELLITERO, Rueil-Malmaison (FR)

(21) Appl. No.: 19/141,264

(22) PCT Filed: Dec. 19, 2023
(86) PCT No.: PCT/FR2023/052049

§ 371 Date: Jun. 19, 2025

(30) Foreign Application Priority Data
Dec. 20, 2022 (FR) 2213922

(51) Int. Cl. B01J 20/18; B01D 9/00; B01D 15/18; B01J 20/28; B01J 20/281; B01J 20/30; C07C 7/13; C07C 7/14

(52) U.S. Cl. CPC B01J 20/186; B01D 9/0045; B01D 15/185; B01J 20/28004; B01J 20/28007; B01J 20/28011; B01J 20/2803; B01J 20/281; B01J 20/3007; B01J 20/3021; B01J 20/3028; B01J 20/3071; B01J 20/3078; B01J 20/3085; C07C 7/13; C07C 7/14; B01D 2009/0086

(57) ABSTRACT

The invention relates to agglomerated zeolitic adsorbents based on Faujasite X type zeolite crystals with a controlled Si/Al molar ratio, exchanged with barium; or based on Faujasite X type zeolite with a controlled Si/Al molar ratio, exchanged with barium and potassium. The invention also relates to the use of the adsorbent in the separation of sugars, polyhydric alcohols, substituted toluene isomers, cresols, or for the recovery of very high purity para-xylene; and to the separation process for separating xylenes that implements the use of the adsorbent.

### TECHNICAL FIELD

The invention relates to agglomerated zeolitic adsorbents based on Faujasite (FAU) type zeolite crystals having a controlled silicon to aluminium, Si/Al, molar ratio, exchanged with barium or barium and potassium and exhibiting selective behaviour for para-xylene present in the aromatic hydrocarbon feed mixture to be separated, the aromatic hydrocarbons comprising 8 carbon atoms containing (C8) isomers.

### PRIOR ART

The use of zeolitic adsorbents consisting of faujasite zeolite (FAU) of type X or Y comprising, in addition to sodium cations, barium and/or potassium and/or strontium ions, alone or in mixtures, in order to selectively adsorb the para-xylene in a mixture of aromatic hydrocarbons, is well known from the prior art.

The patents U.S. Pat. Nos. 3,558,730, 3,558,732, 3,626,020 and 3,663,638 show that aluminosilicate-based zeolitic adsorbents that comprise sodium and barium (U.S. Pat. No. 3,960,774) or comprise sodium, barium and potassium are effective for separating para-xylene present in C8 aromatic fractions (fractions comprising aromatic hydrocarbons having 8 carbon atoms).

One adsorbent preparation method for preparing these adsorbents is described, for example, in the patent U.S. Pat. No. 3,878,127 and consists in treating, in hot sodium hydroxide (soda), agglomerates comprising a zeolite X and binder with a Na₂O/Al₂O₃ ratio strictly less than 0.7 in order to replace the exchangeable cations of the zeolite (such as protons or cations of Group IIA) with sodium, prior to an exchange with barium or barium and potassium, the prior exchange with sodium making it possible for a greater quantity of barium ions or barium and potassium ions to be added to the zeolite structure.

These adsorbents are used as adsorption agents in liquid-phase processes, preferably of the simulated counter-current type, similar to those described in the patent U.S. Pat. No. 2,985,589, which are applicable inter alia to C8 fractions (fractions comprising aromatic hydrocarbons having 8 carbon atoms).

The zeolites of the prior art for the separation of xylenes belong to the Faujasite structural type, first described in the patents U.S. Pat. Nos. 2,882,244 and 3,130,007, which are crystallised silico-aluminates having cages of perfectly determined size and connected in three dimensions.

The patent U.S. Pat. No. 6,884,918 recommends a Faujasite X with a Si/Al atomic ratio between 1.15 and 1.5. The patent U.S. Pat. No. 6,410,815 teaches that zeolitic adsorbents as described in the prior art, but for which Faujasite has a low silica content and has an Si/Al atomic ratio close to 1 (the latter to be referred to as LSX, abbreviation for Low Silica X; or in French: zéolithe X à faible teneur en silice) are advantageously used for the separation of para-xylene.

Zeolite X and low-silica X zeolite therefore both exhibit good performance in terms of para-xylene selectivity, but the synthesis of low-silica X zeolite is rather difficult as compared with the synthesis of zeolite X. Indeed, in order to lower the Si/Al atomic ratio of a Faujasite-type zeolite, it is necessary to increase the consumption of soda used in the zeolite synthesis process. Furthermore, in order to crystallise according to the Faujasite structural type when the Si/Al atomic ratio is 1, it is necessary to add high concentrations of potassium hydroxide (potash) so as to inhibit the formation of zeolite A and obtain only low-silica X zeolite. These high levels of sodium hydroxide and potassium hydroxide consumption increase the cost of manufacturing of this type of zeolite and pose problems in terms of effluent discharges.

In the references listed above, zeolitic adsorbents are in the form of crystals or in the form of agglomerates consisting mainly of zeolite and inert agglomeration binder, generally in a proportion of between 0.1% and 20% by weight.

Since the synthesis of zeolite X and low-silica X zeolites is usually carried out by nucleation and crystallisation of silico-aluminate gels, this produces crystals which are particularly difficult to use on an industrial scale (significant losses of feed mixture during handling) whereas it is preferred to obtain agglomerated forms, for example in the form of granules or grains, which do not present the disadvantages inherent in pulverulent (powdery) materials.

The preparation of these agglomerates is carried out for example by impasting of zeolite crystals with a binder, most often a clay or a mixture of clays, possibly zeolithisable clays, in proportions of the order of 80% to 99.9% by weight of zeolite crystals for 0.1% to 20% by weight of binder, then processed to form granules, grains, beads, pellets or extrudates, and heat-treated at high temperature in order to fire the clay and reactivate the zeolite, it being possible to carry out the barium and/or potassium exchange prior to and/or after agglomeration of the powdered zeolite with the binder.

Zeolitic agglomerates are obtained having a particle size that generally measures a few millimetres and which, if the selection of the binder and the granulation are carried out in accordance with the rules of the state of the art, exhibit a set of satisfactory properties, in particular porosity, mechanical strength and resistance to abrasion.

The French patent FR2925366 describes an agglomerate manufacturing method for manufacturing agglomerates which are based on LSX zeolite crystals with a number-average diameter of less than or equal to 4 μm, with a Si/Al atomic ratio such that (1.00±0.05)≤Si/Al≤1.15 and preferably with a Si/Al atomic ratio=1.00±0.05, of which at least 90% of the exchangeable cationic sites are occupied, either by barium ions alone or by barium ions and potassium ions, in which the mechanical strength as measured by the Shell method series SMS1471-74 that is appropriate for agglomerates having a size that is less than 1.6 mm, is greater than or equal to 2 MPa.

The French patent FR2925367 describes a manufacturing process for manufacturing agglomerated zeolitic adsorbents that comprises a mixture of zeolite X crystals exchanged to at least 90% by barium ions alone or by barium ions and potassium ions, it being possible for the exchangeable sites occupied by the potassium to represent up to one third of the exchangeable sites occupied by the barium+potassium ions (any possible balance generally being made up by alkali or alkaline-earth ions other than barium and potassium); LSX zeolite crystals exchanged to at least 90% by barium ions alone, or by barium ions and potassium ions, it being possible for the exchangeable sites occupied by potassium to represent up to one third of the exchangeable sites occupied by barium+potassium ions (any possible balance generally being made up by alkali or alkaline-earth ions other than barium and potassium); and a binder in a proportion less than or equal to 20% by weight of the total mass of the agglomerate.

The patent U.S. Pat. No. 6,410,815 teaches that the performance levels of the industrial separation process for separating para-xylene depend to a large extent on: the adsorbent, its adsorption capacity, and the degree of selectivity it exhibits for para-xylene in a medium constituted of C₈ aromatic compounds, typically para-xylene, meta-xylene, ortho-xylene, ethylbenzene; as well on the other hand, on the ability of the desorbents, such as toluene and para-diethylbenzene, to desorb the adsorbed para-xylene. The selectivity α_{A/B} of the adsorbent for a component A relative to a compound B is defined as the ratio of the concentrations of the compounds in the adsorbed phase divided by the ratio of the concentrations of the compounds in the non-adsorbed phase at equilibrium:

$${a}_{A / B} = {A}_{ads} / {B}_{ads} \times {B}_{l i q} / {A}_{l i q}$$

where A_{ads} and B_{ads} are the concentrations of compound A and compound B in the adsorbed phase respectively and A_{liq} and B_{liq} are the concentrations of compound A and compound B in the fluid phase.

### SUMMARY OF THE INVENTION

In a surprising manner, it appears that agglomerated zeolitic adsorbents comprising a Faujasite-type zeolite having a controlled silicon to aluminium Si/Al molar ratio and in particular a Si/Al molar ratio such that 1.10≤Si/Al≤1.18, preferably 1.10≤Si/Al≤1.17, even more preferably 1.10≤Si/Al≤1.16, even more preferably 1.11≤Si/Al≤1.16 (to be referred to as MSX, abbreviation for Medium Silica X or in French: zéolithe X à teneur moyenne en silice) that are exchanged to at least 90% by barium ions alone or by barium ions and potassium ions, can advantageously replace the zeolitic adsorbents described in the literature which are based on zeolite X and/or based on zeolite LSX, alone or in mixtures and exchanged with barium or exchanged with barium and potassium. The agglomerated zeolitic adsorbents according to the invention show in particular unexpected levels of performance in terms of selectivity, in a para-xylene separation process. For obvious reasons of ease of implementation and industrial exploitation, the agglomerated zeolitic adsorbents that may be used in the context of the process of the present invention, are used alone, preferably without any other zeolitic adsorbent(s), whether in layers or in mixtures.

The invention relates to an agglomerated zeolitic adsorbent based on MSX zeolite crystals, having an Si/Al atomic ratio such that 1.10≤Si/Al≤1.18, preferably 1.10≤Si/Al≤1.17, more preferably 1.10≤Si/Al≤1.16, more preferably 1.11≤Si/Al≤1.16, of which at least 90%, preferably at least 95% of the exchangeable cationic sites are occupied, either by barium ions alone or by barium ions and potassium ions. More preferably, the invention relates to an agglomerated zeolitic adsorbent based on MSX zeolite crystals, having an Si/Al atomic ratio such that 1.11≤Si/Al<1.15, and more preferably 1.12≤Si/Al<1.15, and advantageously 1.12≤Si/Al≤1.14, of which at least 90%, preferably at least 95% of the exchangeable cationic sites are occupied, either by barium ions alone or by barium ions and potassium ions. Even more preferably, the invention relates to an agglomerated zeolitic adsorbent based on MSX zeolite crystals, having an Si/Al atomic ratio such that 1.11≤Si/Al<1.15, and even more preferably 1.12≤Si/Al<1.15, and advantageously 1.12≤Si/Al≤1.14, of which at least 90%, preferably at least 95%, of the exchangeable cationic sites are occupied, either by barium ions alone or by barium ions and potassium ions.

The micropore volume of the adsorbent, as measured according to the Dubinin method by nitrogen adsorption at 77K after pretreatment at 500° C. for a period of 12 hours under vacuum, may be greater than or equal to 0.200 cm³/g, preferably greater than or equal to 0.220 cm³/g, even more preferably greater than or equal to 0.225 cm³/g, in an even more preferable manner greater than or equal to 0.250 cm³/g.

The exchangeable sites occupied by the potassium may represent up to ⅓ of the exchangeable sites occupied by the barium+potassium ions, with any possible balance being generally made up by alkali or alkaline-earth ions other than barium and potassium.

The adsorbent may comprise an inert binder in a proportion that is less than or equal to 20% by weight, preferably 15% by weight, of the total mass of the adsorbent.

The adsorbent may have a size distribution such that the number-average diameter is between 0.4 mm and 2.0 mm, preferably between 0.4 mm and 0.8 mm.

The mechanical strength of the adsorbent, as measured by the Shell method series SMS1471-74 that is appropriate for agglomerates having a size that is less than 1.6 mm, is advantageously greater than or equal to 2 MPa, preferably greater than or equal to 2.5 MPa.

The loss on ignition as measured at 900° C. is advantageously less than or equal to 7.7%, preferably between 0 and 7.7%, in a preferable manner between 3.0% and 7.7%, in an even more preferable manner between 3.5% and 6.5%, and advantageously between 4.5% and 6.0%, inclusive of limits.

The MSX zeolite crystals may have a number average diameter of between 0.01 μm and 5 μm, preferably between 0.05 μm and 5 μm, in a highly preferable manner between 0.1 μm and 4 μm, in an even more preferable manner between 0.1 μm and 3 μm, and in an even more preferable manner between 0.1 μm and 2 μm.

The invention also relates to a separation process for separating sugars, polyhydric alcohols, substituted toluene isomers, cresols, or for recovering para-xylene, by means of an agglomerated zeolitic adsorbent according to any of the variants described, in the presence of a desorbent, in liquid phase or in gas phase.

The process may be a para-xylene recovery process for recovering para-xylene from fractions of C₈ aromatic isomers, by adsorption of para-xylene by means of the said agglomerated zeolitic adsorbent, in the presence of a desorbent, in liquid phase or in gas phase.

The recovery process for recovering para-xylene may be implemented by means of a simulated moving bed process, of the simulated co-current or simulated counter-current type.

The process may be a production process for producing high purity, high productivity para-xylene from a feed mixture of aromatic hydrocarbons comprising 8 carbon atoms containing (C8) isomers, the process including the following steps:
a) a contacting step for bringing the feed mixture into contact with a bed of agglomerated zeolitic adsorbent, in a manner so as to preferentially adsorb the para-xylene;
b) a contacting step, under desorption conditions, for bringing the adsorbent bed into contact with a desorbent, which is preferably either toluene, or para-diethylbenzene;
c) a withdrawal step for withdrawing from the adsorbent bed a stream containing the desorbent and the least selectively adsorbed products of the feed mixture;
d) a withdrawal step for withdrawing from the adsorbent bed a stream containing the desorbent and the para-xylene;
e) a separation step for separating the stream obtained from the step c) into a first stream containing the desorbent and a second stream containing the least selectively adsorbed products of the feed mixture; and
f) a separation step for separating the stream obtained from the step d) into a first stream containing the desorbent and a second stream containing para-xylene having a purity level greater than or equal to 75%.

The said process may in addition include the following steps:
g) a crystallisation step in a crystalliser, consisting of the crystallisation of the para-xylene resulting from the step f), thereby making it possible to obtain, on the one hand, the para-xylene crystals soaked in the mother liquor thereof; and on the other hand, a mother liquor which may in part, or even in its entirety, be recycled as a mixture with the fresh feed mixture at the inlet of the simulated moving bed adsorption unit; and
h) a washing step for washing the crystals resulting from the step g), at the end of which para-xylene is recovered having a purity of at least 99.7%, and in a preferable manner of at least 99.8%.

More generally, the invention finally relates to the use of an agglomerated zeolitic adsorbent based on MSX zeolite crystals having an Si/Al atomic ratio such that 1.10≤Si/Al≤ 1.18, preferably 1.10≤Si/Al≤1.17, even more preferably 1.10≤Si/Al≤1.16, even more preferably 1.11≤Si/Al≤1.16, of which at least 90% of the exchangeable cationic sites are occupied, either by barium ions alone or by barium ions and potassium ions, for the separation of sugars, polyhydric alcohols, substituted toluene isomers, cresols, or for the recovery of para-xylene, in the presence of a desorbent, which is preferably either toluene or para-diethylbenzene, in liquid phase or in gas phase.

This use may relate to the recovery of para-xylene from fractions of C8 aromatic isomers, by adsorption of para-xylene in a simulated moving bed reactor, of the simulated co-current, or simulated counter-current type.

### DESCRIPTION OF EMBODIMENTS

The object of the present invention relates to zeolitic adsorbents that may be used in particular for the separation of para-xylene from a mixture of C₈ aromatic compounds that present excellent performance levels, in particular in terms of selectivity for the para-xylene, the said adsorbents being particularly suitable for use in a separation process for separating para-xylene in the liquid phase, and most particularly in a high productivity separation process for separating para-xylene in the liquid phase, being preferably of the simulated counter-current type.

The agglomerated zeolitic adsorbents according to the present invention comprise an MSX zeolite having a Si/Al atomic ratio such that 1.10≤Si/Al≤1.18, preferably 1.10≤Si/Al≤1.17, even more preferably 1.10≤Si/Al≤1.16, even more preferably 1.11≤Si/Al≤1.16, of which the lower values reflect the analytical uncertainties in the measurement of this ratio, and the higher values, either the same analytical uncertainty, or a tolerable deviation in the purity of the product, exchanged to at least 90% by barium ions alone or by barium ions and potassium ions, it being possible for the exchangeable sites occupied by the potassium to represent up to ⅓ of the exchangeable sites occupied by the barium+potassium ions (any possible balance being generally made up by alkali or alkaline-earth ions other than barium and potassium); the mechanical strength of the agglomerated zeolitic adsorbents according to the invention is measured by the Shell method series SMS1471-74 that is appropriate for agglomerates having a size that is less than 1.6 mm, and is advantageously greater than or equal to 2 MPa.

Advantageously, the zeolitic adsorbents according to the invention may comprise a binder in a proportion of less than or equal to 20% by weight, preferably 15% by weight, of the total mass of the agglomerate. This binder may contain one or more zeolithisable clay(s), and preferably at least 80% by weight of zeolithisable clay(s), and optionally one or more additive(s).

The number average diameter of the zeolite crystals in the zeolitic adsorbents according to the invention is advantageously between 0.01 μm and 5 μm, preferably between 0.05 μm and 5 μm, in a highly preferable manner between 0.1 μm and 4 μm, in an even more preferable manner between 0.1 μm and 3 μm and, in an even more preferable manner between 0.1 μm and 2 μm

In a general manner, the zeolitic adsorbents according to the invention have a volume average diameter of 0.4 mm to 2 mm, and in particular between 0.4 mm and 0.8 mm.

In this document, the term “number average diameter” or indeed “size”, is used for zeolite crystals and for zeolitic agglomerates. Accuracy of measurement is of the order of 3%.

The invention also relates to a preparation process for preparing zeolitic adsorbents according to the invention. The agglomerated zeolitic adsorbents according to the invention may be prepared according to a preparation process which includes the following steps:
a/agglomeration of zeolite MSX crystals with a binder containing at least 80% by weight of zeolithisable clay and optionally additives, and subsequently the forming, followed by drying and calcination; and
b/possibly, zeolithisation of the binder by the action of a basic alkaline solution;
c/replacement of at least 90% of the exchangeable sites of the MSX zeolite with barium, followed by washing and drying of the product thus treated;
d/possibly, replacement of up to 33% of the exchangeable sites of zeolite X with potassium, followed by washing and drying of the product thus treated,
e/activation.

The agglomeration and the forming (step a/) may be carried out in accordance with any of the techniques known to the person skilled in the art, such as extrusion, compacting and agglomeration. The agglomeration binder represents a proportion of less than or equal to 20% by weight, preferably 15% by weight, of the total mass of the agglomerate.

According to one preferred embodiment, the zeolite MSX crystals have a diameter of less than or equal to 5 μm, in a preferable manner between 0.01 μm and 5 μm, preferably between 0.05 μm and 5 μm, in a highly preferable manner between 0.1 μm and 4 μm, in an even more preferable manner between 0.1 μm and 3 μm and, in an even more preferable manner between 0.1 μm and 2 μm.

At the end of step a/, the finest agglomerate particles may be removed by means of cycloning and/or screening, and/or the excessively large particles may be removed by means of screening or crushing, for example in the case of extrudates.

The agglomeration binder used in implementation of the step a/contains at least 80% by weight of zeolithisable clay and may also contain other mineral binders such as bentonite or attapulgite. The term “zeolithisable clay” is used to refer to a clay or a mixture of clays which are capable of being transformed into a zeolithic material by the action of a basic/alkaline solution. Zeolithisable clays generally belong to the family of kaolins, kaolinites, nacrites, dickites, halloysites and/or metakaolins. The commonly used clay is kaolin.

During the step a/, in addition to the zeolite MSX crystals and the binder, one or more additive(s) may also be used, for example additives intended to facilitate agglomeration or to enhance the hardening of the agglomerates formed.

The additives that may possibly be used in the step a) may include a source of silica of any type known to the person skilled in the art, who specialises in the synthesis of zeolites, for example colloidal silica, diatomaceous earth, perlite, ash from calcination (that is, fly ash), sand, or any other form of solid silica.

The zeolite MSX crystals used in implementation in the step a/may be derived from the synthesis of sodium-exchanged zeolite MSX crystals, although it would not be a departure from the scope of the invention to use crystals which have undergone one or more cation exchanges, between the synthesis in the NaMSX form and the implementation thereof in the step a/.

The calcination which follows the drying is carried out at a temperature generally between 500° C. and 600° C. According to one preferred embodiment, the step b/of zeolithisation is carried out.

When the step b/of zeolithisation is carried out, the transformation of at least 50% of the inert zeolithisable binder into zeolithic material is obtained; it should be noted that the purpose of zeolithisation is in particular to increase the mechanical strength of the agglomerated zeolitic adsorbents. The zeolithisation may be carried out by immersing the agglomerate in a basic/alkaline solution, which is generally aqueous, for example an aqueous solution of sodium hydroxide and/or potassium hydroxide, the concentration of which is preferably greater than 0.5 M. It is preferable for the process to be carried out hot (temperature above ambient temperature), typically at temperatures of the order of 80° C. to 100° C., in order to enhance the process kinetics and to reduce the immersion time periods to less than 8 hours; however, it would not be a departure from the scope of the invention to carry out the operation at lower temperatures and for longer immersion time periods. According to this operating method, it is possible to readily achieve zeolithisation (i.e. the conversion of the adsorption-inert binder into adsorption-active material) of at least 50% by weight of the binder. The material is then washed with water before subsequently being dried.

The step c/of exchanging the cations of the zeolite with barium takes place by bringing the agglomerates obtained from the step b/(or d/) into contact with a barium salt, such as BaCl₂, in aqueous solution at a temperature between ambient temperature and 100° C., and preferably between 80° C. and 100° C. In order to rapidly obtain a high degree of barium exchange, i.e. greater than 90%, it is preferable to operate with a large excess of barium in relation to the cations of the zeolite which it is desired to exchange, typically such that the BaO/Al₂O₃ ratio is of the order of 10 to 12, and by carrying out successive exchanges in such a manner as to achieve the targeted minimum level of exchange of at least 90%, and preferably at least 95%. Throughout the text, the levels of exchange are calculated in equivalents and not in terms of molarity.

The optional exchange with potassium (step d/) may be carried out prior to and/or after the exchange with barium (step c/), and/or simultaneously using a solution containing the barium ions and the potassium ions. As previously indicated, it is also possible to agglomerate in the step a/the zeolite MSX crystals already containing potassium ions (by pre-exchanging the NaMSX zeolite with potassium ions before the step a/) and to dispense (or not) with the step d/.

The purpose of the activation (step e/), the last step in the process for obtaining the adsorbents according to the invention, is to fix the water content and the loss on ignition of the adsorbent within optimum limits. This is generally done by means of thermal activation, which is preferably carried at a temperature of between 20° and 300° C. for a certain period of time depending on the desired water content and the desired loss on ignition, typically from 1 to 6 hours.

In one embodiment of the present invention, the loss on ignition of the agglomerated zeolitic adsorbent according to the invention, as measured at 900° C. in accordance with French standard NF EN 196-2, is less than or equal to 7.7%, preferably between 0 and 7.7%, in a preferable manner between 3.0% and 7.7%, in an even more preferable manner between 3.5% and 6.5%, and advantageously between 4.5% and 6.0%, inclusive of the limits.

The agglomerates resulting from the step e/, whether they are in the form of beads or extrudates, generally have a number-average diameter ranging from 0.4 mm to 2.0 mm, and in particular between 0.4 mm and 0.8 mm.

In a general manner, the number-average diameter of the zeolite crystals in these zeolitic adsorbents resulting from the steps a/to e/is between 0.01 μm and 5 μm, preferably between 0.05 μm and 5 μm, in a highly preferable manner between 0.1 μm and 4 μm, in an even more preferable manner between 0.1 μm and 3 μm and, in an even more preferable manner between 0.1 μm and 2 μm.

The invention also relates to the uses of at least one zeolitic adsorbent according to any one of the variants described, as adsorption agents capable of advantageously replacing the adsorption agents described in the literature based on zeolite X or based on zeolite LSX, exchanged with barium or exchanged with barium and potassium, and in particular in the following uses:
separation of C₈ aromatic isomers and in particular xylenes;
separation of sugars;
separation of polyhydric alcohols;
separation of substituted toluene isomers, such as nitrotoluene, diethyltoluene, toluenediamine,
separation of cresols;
separation of dichlorobenzenes.

The invention relates in particular to a recovery process for recovering para-xylene from fractions of C₈ aromatic isomers that consists of using as an adsorption agent for adsorbing para-xylene, a zeolitic adsorbent according to the invention used in implementation in liquid phase processes, but also in the gas phase processes.

The invention particularly relates to a production process for producing high purity and high productivity para-xylene from a feed mixture of aromatic hydrocarbons comprising 8 carbon atoms containing (C8) isomers, the process including the following steps:
a) a contacting step, under appropriate adsorption conditions, for bringing the feed mixture into contact with an adsorbent bed according to the invention, in a manner so as to preferentially adsorb the para-xylene;
b) a contacting step, under desorption conditions, for bringing the adsorbent bed into contact with a desorbent, which is preferably either toluene, or para-diethylbenzene,
c) a withdrawal step for withdrawing from the adsorbent bed a stream containing the desorbent and the least selectively adsorbed products of the feed mixture;
d) a withdrawal step for withdrawing from the adsorbent bed a stream containing the desorbent and the para-xylene;
e) a separation step for separating the stream obtained from the step c) into a first stream containing the desorbent and a second stream containing the least selectively adsorbed products of the feed mixture; and
f) a separation step for separating the stream obtained from the step d) into a first stream containing the desorbent and a second stream containing para-xylene having a purity level greater than or equal to 75%, and preferably greater than or equal to 99.7%.

The process may also optionally include the following steps:
g) a crystallisation step in a crystalliser, consisting of the crystallisation of the para-xylene resulting from the step f), thereby making it possible to obtain, on the one hand, the para-xylene crystals soaked in the mother liquor thereof; and on the other hand, a mother liquor which may in part, or even in its entirety, be recycled as a mixture with the fresh feed mixture at the inlet of the simulated moving bed adsorption unit; and
h) a washing step for washing the crystals resulting from the step g), at the end of which para-xylene is recovered having a purity of at least 99.7%, and in a preferable manner of at least 99.8%.

The desired product may thus be separated by preparative adsorption liquid chromatography (in “batch” mode), advantageously by means of simulated moving bed liquid chromatography, that is to say of the simulated counter-current or simulated co-current type, and more particularly simulated counter-current type.

Chromatographic separation based on simulated counter-current moving bed chromatography, is well known in the state of the art. As a general rule, a simulated moving bed separation unit comprises at least one adsorption column containing a plurality of beds of an adsorbent, which are interconnected in a closed loop. The simulated moving bed separation unit includes at least three chromatographic zones, and possibly four or five, each of these zones being constituted by at least one bed or a portion of column and comprised between two successive feed or withdrawal points.

Typically, at least one feed mixture to be fractionated and one desorbent (sometimes referred to as the eluent) are fed and at least one raffinate and one extract are withdrawn. The feed and draw-off points are modified over time, typically shifted towards the bottom of a bed and in a synchronous manner.

By definition, each operating zone is denoted by a number:
Zone 1=zone of desorption for the desired product (contained in the extract) between injection of the desorbent and extraction of the extract;
Zone 2=zone of desorption for the raffinate compounds, comprised between the withdrawal of the extract and the injection of the feed mixture to be fractionated;
Zone 3=zone of adsorption of the desired product, between injection of the feed mixture and withdrawal of the raffinate, and;
Zone 4 situated between the withdrawal of the raffinate and the injection of the desorbent.

The operating conditions for an industrial adsorption unit of the simulated counter-current type are in general as follows:
number of beds: 6 to 30
number of zones: at least 4
temperature: 100 to 250° C., preferably 150 to 190° C.
pressure: between the bubble pressure of xylenes at a temperature of the process and 3 MPa;
ratio of desorbent rate to feed rate: 0.7 to 2.5 (for example 0.9 to 1.8 for a single adsorption unit (“stand-alone” unit as per the accepted terminology) and 0.7 to 1.4 for an adsorption unit combined with a crystallisation unit);
recycling rate: from 2.5 to 12, preferably 3.5 to 6. The recycling rate is defined as the ratio of the average flow rate flowing through the different beds of the adsorber to the flow rate of injection of feed into this adsorber.

Further reference may be made to the teachings in the patents U.S. Pat. Nos. 2,985,589, 5,284,992 and 5,629,467.

The operating conditions of an industrial simulated co-current adsorption unit are in general the same as those operating in simulated counter-current mode with the exception of the recycling rate, which is generally between 0.8 and 7. Further reference may be made to the teachings in the patents U.S. Pat. Nos. 4,402,832 and 4,498,991.

The desorption solvent may be a desorbent having a boiling point that is lower than that of the feed, such as toluene, but also a desorbent having a boiling point that is higher than that of the feed, such as para-diethylbenzene (PDEB). The selectivity of the adsorbents according to the invention for the adsorption of para-xylene contained in C₈ aromatic fractions is optimal when the loss on ignition thereof as measured at 900° C. is generally less than or equal to 7.7%, preferably between 0 and 7.7%, in a preferable manner between 3.0% and 7.7%, in a more preferable manner between 3.5% and 6.5%, and advantageously between 4.5% and 6.0%, inclusive of limits.

One of the techniques of choice for characterising the adsorption of molecules in liquid phase on a porous solid is that of breakthrough. In his work “Principles of Adsorption and Adsorption Processes”, Ruthven defines the technique of breakthrough curves as a way of studying the injection of a range of adsorbable constituents

The present invention will now be described using the examples that follow, which are intended to illustrate certain embodiments of the invention, without however limiting the scope of the said invention, as claimed in the appended claims.

### Analytical Techniques

### Identification of Zeolitic Phases

The zeolite MSX in the zeolitic adsorbents of the invention is identified by X-ray diffraction analysis, known to the person skilled in the art by the initialism DRX. This analysis is carried out on a DRX D8 Advance device from the company Bruker. The identification of phases is carried out using Bruker's EVA software and databases known to the person skilled in the art that contain a large number of diffractograms, such as the database ICCD PDF-2 release 2011.

### Si/Al Molar Ratio and Rate of Exchange

The measurement of the Si/Al molar ratio and the rate of exchange is carried out by any of the analytical chemical analysis techniques known to the person skilled in the art.

Among these techniques, it is worth mentioning the X-ray fluorescence chemical analysis technique as described in French standard NF EN ISO 12677:2011 on a wavelength dispersive X-ray spectrometer (WDXRF), for example Tiger S8 from the company Bruker.

X-ray fluorescence (XRF) spectrometry is a non-destructive spectral analytical technique that exploits the photoluminescence of atoms in the X-ray range, in order to determine the elemental composition of a sample. The excitation of atoms, generally by a beam of X-rays or by bombardment with electrons, generates specific radiation once the atom has returned to its fundamental state. The advantage offered by the X-ray fluorescence spectrum is that it depends very little on the chemical combination of the element, which thus provides for precise determination, both in quantitative and qualitative terms. In a conventional manner, following calibration for each oxide, a measurement uncertainty of less than 0.4% by weight is obtained.

These elementary chemical analyses make it possible both to verify the Si/Al molar ratio of the initial zeolite as well as to ascertain the quality of the ion exchange described in the step c) and optional step d).

The quality of ion exchange is related to the number of moles of sodium oxide, Na₂O, remaining in the agglomerated zeolitic adsorbent after exchange. More precisely, the degree of exchange with barium ions is estimated by assessing the ratio between the number of moles of barium oxide, BaO, and the number of moles of the composite whole (BaO+Na₂O). Likewise, the degree of exchange with barium and potassium ions is estimated by assessing the ratio between the number of moles of the barium oxide+potassium oxide (BaO+K₂O) and the number of moles of the composite whole (BaO+K₂O+Na₂O). It should be noted that the content levels of different oxides are given as a percentage by weight relative to the total weight of the anhydrous zeolitic adsorbent.

The Si/Al molar ratio of the zeolite present in the agglomerated zeolitic adsorbent is measured by solid-state Nuclear Magnetic Resonance (NMR) spectroscopy of silicon.

### Particle Size Distribution (Granulometry) of the Zeolite Crystals:

The estimation of the number average diameter of the zeolite crystals used in step a) and of the zeolite crystals contained in the agglomerates is carried out by observation with a scanning electron microscope (SEM).

In order to estimate the size of the zeolite particles (i.e. crystals) found in the samples, a series of images is captured at a magnification of at least 5,000. The diameter of at least 200 particles is then measured with the help of dedicated software, such as Smile View developed by LoGraMi. The measurement accuracy is of the order of 3%. Measurement of the histogram constituted on the basis of these diameter measurements also enables the standard deviation σ of its distribution to be determined.

The observation of the zeolite crystals using a scanning electron microscope (SEM) can also be used to distinguish the crystalline structures of the zeolites (LSX, MSX, X).

### Particle Size Distribution (Granulometry) of the Zeolitic Adsorbents:

The number-average diameter of the zeolitic adsorbents obtained as a result of the step a) of agglomeration and forming is determined by analysing the particle size distribution of an agglomerate sample by means of imaging in accordance with the standard ISO 13322-2:2006, making use of a conveyor belt that enables the sample to pass in front of the camera lens.

The number average diameter is then calculated based on the particle size distribution in accordance with the standard ISO 9276-2:2001. In this document, the terms “number average diameter” or “size” are used for the zeolitic agglomerates. The accuracy is of the order of 0.01 mm for the size range of agglomerates of the invention.

### Micropore Volume:

The crystallinity of the agglomerates is also evaluated by measuring their micropore volume by comparing the latter to that of an appropriate reference (100% crystalline zeolite under identical cationic treatment conditions or theoretical zeolite). This micropore volume is determined based on the measurement of the adsorption isotherm of gas, such as nitrogen, at its liquefaction temperature.

Prior to adsorption, the zeolitic adsorbent is degassed between 300° C. and 450° C. for a period of between 9 hours and 16 hours, under vacuum (P<6.7.10⁻⁴ Pa). The measurement of the nitrogen adsorption isotherm at 77 K is then performed on an apparatus of the type ASAP 2020 M from Micromeritics, by taking at least 35 measurement points at relative pressures with a P/P₀ ratio of between 0.002 and 1.

### Loss on Ignition of the Zeolitic Adsorbents:

The loss on ignition is determined in an oxidizing atmosphere, by means of calcination of the sample in air at a temperature of 900° C.±25° C., following the operating procedure described in the French standard NF EN 196-2 (April 2006). The standard deviation of measurement is less than 0.1%.

### EXAMPLES

### Example 1: (Comparative): Preparation of a BaLSX Type Adsorbent Comprising a Zeolitised Binder

In this example, an adsorbent according to the prior art is produced and tested.

840 g (expressed as calcined equivalent) of zeolite LSX crystals with a Si/Al ratio of 1.01, and 160 g of kaolin (expressed as calcined equivalent) are intimately mixed and agglomerated with the appropriate quantity of water so as to enable agglomeration by extrusion to take place. The extrudates are dried, then crushed in a manner such as to recover grains that have an equivalent diameter of 0.7 mm, after which they are calcined at 550° C. under a stream of nitrogen for a period of 2 hours.

200 g of the granules thus obtained are placed in a glass reactor fitted with a double jacket regulated at a temperature of 95±1° C.; this is followed by the addition of 700 mL of an aqueous sodium hydroxide solution at a concentration of 220 g/L, after which the reaction medium is left under agitation for a period of 3 hours.

The granules are then subjected to washing in 4 successive wash operations using water, followed by draining of the reactor. The effectiveness of the wash is ascertained by measuring the final pH of the wash water, which should be between 10 and 10.5.

A barium exchange is then carried out under operating conditions identical to those in Example 1, followed by a wash operation, then drying at 80° C. for a period of 2 hours, which is finally followed by activation at 200° C. for a period of 2 hours under a stream of nitrogen.

The degree of barium exchange for this adsorbent is 97%. The micropore volume as measured according to the Dubinin method by nitrogen adsorption at 77 K after pre-treatment at 500° C. for a period of 12 hours under vacuum, is 0.235 cm³/g.

The mechanical strength is also measured according to the method presented in the description of the invention. The pressure needed in order to obtain 0.5% fines is 2.70 MPa.

### Example 2 (Comparative): Preparation of a BaX Type Adsorbent Comprising a Zeolitised Binder

In this example, an adsorbent according to the prior art is produced and tested.

900 g (expressed as calcined equivalent) of zeolite X crystals, having a Si/Al ratio of 1.25 and an average crystal size of 1.6 μm, are agglomerated by mixing them intimately with 170 g of kaolin (expressed as calcined equivalent), 70 g of colloidal silica sold under the trade name Klebosol® 30 (containing 30% by weight of SiO₂ and 0.5% of Na₂O), and with the appropriate quantity of water required for forming the agglomerates by extrusion. The extrudates are dried, then crushed in a manner such as to recover agglomerates that have an equivalent diameter of 0.7 mm, after which they are activated at a temperature of 550° C. for a period of 2 hours under a stream of nitrogen.

200 g of the granules thus obtained are placed in a glass reactor fitted with a double jacket regulated at a temperature of 100±1° C.; this is followed by the addition of 1.5 L of an aqueous sodium hydroxide solution at a concentration of 100 g/L, after which the reaction medium is left under agitation for a period of 3 hours. The granules are then subjected to washing in 3 successive wash operations using water, followed by draining of the reactor. The effectiveness of the wash is ascertained by measuring the final pH of the wash water, which should be between 10 and 10.5.

A barium exchange is then carried out under operating conditions identical to those in Example 1, followed by a wash operation, then drying at 80° C. for a period of 2 hours, which is finally followed by activation at 200° C. for a period of 2 hours under a stream of nitrogen.

The degree of barium exchange for this adsorbent is 95%.

The micropore volume as measured according to the Dubinin method by nitrogen adsorption at 77 K after pre-treatment at 500° C. for a period of 12 hours under vacuum, is 0.256 cm³/g.

The mechanical strength is also measured according to the method presented in the description of the invention. The pressure needed in order to obtain 0.5% fines is 2.50 MPa.

### Example 3 (Comparative): 50:50 Weight Mixture of Samples from Examples 1 and 2 (BaLSX Type Zeolitic Adsorbent and BaX Type Zeolitic Adsorbent)

The samples of Examples 1 and 2 in 50:50 weight proportions are mixed mechanically.

The apparent Si/Al molar ratio of this mixture is 1.13.

Example 4 (according to the invention): Preparation of a BaMSX type adsorbent comprising a zeolitised binder

Preparation is undertaken of the BaMSX crystals (Si/Al=1.14) according to Table 4, Example 27, of the patent U.S. Pat. No. 6,596,256.

Analysis of the size of the zeolite crystals is carried out by means of scanning electron microscopy. The average crystal size is 2.8 μm.

840 g (expressed as calcined equivalent) of these zeolite MSX crystals are intimately mixed and agglomerated with 170 g of kaolin (expressed as calcined equivalent), 40 g of colloidal silica sold under the trade name Klebosol® 30 (containing 30% by weight of SiO₂ and 0.5% of Na₂O), and with the appropriate quantity of water so as to enable agglomeration by extrusion to take place. The extrudates are dried, then crushed in a manner such as to recover grains that have an equivalent diameter of 0.7 mm, after which they are calcined at 600° C. under a stream of nitrogen for a period of 2 hours.

200 g of the granules thus obtained are placed in a glass reactor fitted with a double jacket regulated at a temperature of 95+1° C.; this is followed by the addition of 700 ml of an aqueous sodium hydroxide solution at a concentration of 170 g/L, after which the reaction medium is left under agitation for a period of 3 hours. The granules are then subjected to washing in 3 successive wash operations using water, followed by draining of the reactor.

The effectiveness of the wash is ascertained by measuring the final pH of the wash water, which should be between 10 and 10.5.

A barium exchange is then carried out under operating conditions identical to those in Example 1, followed by a wash operation, then drying at 80° C. for a period of 2 hours, which is finally followed by activation at 200° C. for a period of 2 hours under a stream of nitrogen.

The degree of barium exchange for this adsorbent is 97%. The micropore volume as measured according to the Dubinin method by nitrogen adsorption at 77 K after pre-treatment at 500° C. for a period of 12 hours under vacuum, is 0.255 cm³/g.

The mechanical strength is also measured according to the method presented in the description of the invention. The pressure needed in order to obtain 0.5% fines is 2.60 MPa.

An agglomerated zeolitic adsorbent of BaMSX is obtained according to the invention.

### Example 5: Breakthrough Test (Frontal Chromatography)

The Loss On Ignition (LIO) is adjusted for each sample to a value of 6.0%.

A breakthrough test (frontal chromatography) is then carried out on these adsorbents in order to determine their effectiveness. The amount of adsorbent used for this test is approximately 82 g.

The operating method as well as the composition of the feed are identical to those of Example 1.

The operating method for obtaining the breakthrough curves is as follows:
Filling of the column with the screen or sieve and placing of the column in the test bench.
Filling with the solvent at ambient temperature.
Progressive rise in the adsorption temperature under a stream of solvent (5 cm³/min).
Injection of solvent at 10 cm³/min when the adsorption temperature is reached
Solvent/feed permutation for injecting the feed (10 cm³/min).
Injection of the feed is then maintained for a time long enough to reach thermodynamic equilibrium; and
Collection and analysis of the breakthrough effluent.

The pressure is sufficient for the feed to remain in the liquid phase, i.e. 1 MPa. The adsorption temperature is 175° C.

The composition of the feed is as follows:

Para-xylene: 45% by weight

Meta-xylene: 45% by weight

Iso-octane: 10% by weight (the latter is used as tracer for estimating the non-selective volumes and is not involved in the separation)

The breakthrough results are summarised in Table 1.

TABLE 1

| | Si/Al | Selectivity α_{PX/MX (1)} |
|---|---|---|
| Example 1 comparative | 1.00 | 3.75 |
| Example 2 comparative | 1.25 | 3.43 |
| Example 3 comparative | 1.13 | 3.55 |
| Example 4 (according to the | 1.14 | 4.10 |
| invention) | | |

₍₁₎ PX: para-xylene, MX: meta-xylene

In a surprising manner, the zeolitic adsorbents according to the invention exhibit a selectivity for para-xylene over meta-xylene that is far greater than the values measured with the zeolitic adsorbents of the prior art.

1. An agglomerated zeolitic adsorbent based on MSX zeolite crystals, having an Si/Al atomic ratio such that 1.10≤Si/Al≤1.18, preferably 1.10≤Si/Al≤1.17, more preferably 1.10≤Si/Al≤1.16, more preferably 1.11≤Si/Al≤1.16, of which at least 90%, preferably at least 95% of the exchangeable cationic sites are occupied, either by barium ions alone or by barium ions and potassium ions.

2. An agglomerated zeolitic adsorbent according to claim 1, of which the micropore volume as measured according to the Dubinin method by nitrogen adsorption at 77K after pretreatment at 500° C. for a period of 12 hours under vacuum, is greater than or equal to 0.200 cm³/g, preferably greater than or equal to 0.220 cm³/g, even more preferably greater than or equal to 0.225 cm³/g, in an even more preferable manner greater than or equal to 0.250 cm³/g.

3. An agglomerated zeolitic adsorbent according to claim 1, of which the exchangeable sites occupied by the potassium represent up to ⅓ of the exchangeable sites occupied by the barium+potassium ions, with any possible balance being generally made up by alkali or alkaline-earth ions other than barium and potassium.

4. An agglomerated zeolitic adsorbent according to claim 1, comprising an inert binder in a proportion that is less than or equal to 20% by weight, preferably 15% by weight, of the total mass of the agglomerate.

5. An agglomerated zeolitic adsorbent according to one of the preceding claims, that has a size distribution such that the number-average diameter is between 0.4 mm and 2.0 mm, preferably between 0.4 mm and 0.8 mm.

6. An agglomerated zeolitic adsorbent according to one of the preceding claims, of which the mechanical strength, as measured by the Shell method series SMS1471-74 that is appropriate for agglomerates having a size that is less than 1.6 mm, is greater than or equal to 2 MPa, preferably greater than or equal to 2.5 MPa.

7. An agglomerated zeolitic adsorbent according to one of the preceding claims, whereof the loss on ignition as measured at 900° C. is less than or equal to 7.7%, preferably between 0 and 7.7%, in a preferable manner between 3.0% and 7.7%, in an even more preferable manner between 3.5% and 6.5%, and advantageously between 4.5% and 6.0%, inclusive of limits.

8. An agglomerated zeolitic adsorbent according to one of the preceding claims, in which the MSX zeolite crystals have a number average diameter of between 0.01 μm and 5 μm, preferably between 0.05 μm and 5 μm, in a highly preferable manner between 0.1 μm and 4 μm, in an even more preferable manner between 0.1 μm and 3 μm, and in an even more preferable manner between 0.1 μm and 2 μm.

9. A separation process for separating sugars, polyhydric alcohols, substituted toluene isomers, cresols, or for recovering para-xylene, by means of an agglomerated zeolitic adsorbent according to any one of claims 1 to 8, in the presence of a desorbent, in liquid phase or in gas phase.

10. A para-xylene recovery process according to claim 9, for recovering para-xylene from fractions of C₈ aromatic isomers, by adsorption of para-xylene by means of the said agglomerated zeolitic adsorbent, in the presence of a desorbent, in liquid phase or in gas phase.

11. A recovery process according to any one of claim 9 or 10, implemented by means of a simulated moving bed process, of the simulated co-current or simulated counter-current type.

12. A production process according to one of claims 9 to 11, for producing high purity and high productivity para-xylene from a feed mixture of aromatic hydrocarbons comprising 8 carbon atoms containing (C8) isomers, the process including the following steps:
a) a contacting step for bringing the feed mixture into contact with a bed of agglomerated zeolitic adsorbent, in a manner so as to preferentially adsorb the para-xylene;
b) a contacting step, under desorption conditions, for bringing the adsorbent bed into contact with a desorbent, which is preferably either toluene, or para-diethylbenzene;
c) a withdrawal step for withdrawing from the adsorbent bed a stream containing the desorbent and the least selectively adsorbed products of the feed mixture;
d) a withdrawal step for withdrawing from the adsorbent bed a stream containing the desorbent and the para-xylene;
e) a separation step for separating the stream obtained from the step c) into a first stream containing the desorbent and a second stream containing the least selectively adsorbed products of the feed mixture; and
f) a separation step for separating the stream obtained from the step d) into a first stream containing the desorbent and a second stream containing para-xylene having a purity level greater than or equal to 75%.

13. A process according to claim 12 that in addition includes:
g) a crystallisation step in a crystalliser, consisting of the crystallisation of the para-xylene resulting from the step f), thereby making it possible to obtain, on the one hand, the para-xylene crystals soaked in the mother liquor thereof; and on the other hand, a mother liquor which may in part, or even in its entirety, be recycled as a mixture with the fresh feed mixture at the inlet of the simulated moving bed adsorption unit; and
h) a washing step for washing the crystals resulting from the step g), at the end of which para-xylene is recovered having a purity of at least 99.7%, and in a preferable manner of at least 99.8%.

14. The use of an agglomerated zeolitic adsorbent based on MSX zeolite crystals having an Si/Al atomic ratio such that 1.10≤Si/Al≤1.18, preferably 1.10≤Si/Al≤1.17, even more preferably 1.10≤Si/Al≤1.16, even more preferably 1.11≤Si/Al≤1.16, of which at least 90% of the exchangeable cationic sites are occupied, either by barium ions alone or by barium ions and potassium ions, for the separation of sugars, polyhydric alcohols, substituted toluene isomers, cresols, or for the recovery of para-xylene, in the presence of a desorbent, which is preferably either toluene or para-diethylbenzene, in liquid phase or in gas phase.

15. The use of an agglomerated zeolitic adsorbent according to claim 14, the recovery of para-xylene from fractions of C8 aromatic isomers, by adsorption of para-xylene in a simulated moving bed reactor, of the simulated co-current, or simulated counter-current type
